(19) United States
(12) Patent Application Publication
(10) Pub. No.: US 2025/0137986 A1
(43) Pub. Date: May 1, 2025

(54) LASER WELDED BLANK WELD SEAM SAFETY DETERMINATION METHOD

(71) Applicant: FCA US LLC, Auburn Hills, MI (US)

(72) Inventors: Dajun Zhou, Troy, MI (US); Chunlei Wang, Rochester Hills, MI (US); Kaiping Li, Shelby Township, MI (US); Robert D Miller, Lake Orion, MI (US); Yongjun Zhou, Rochester Hills, MI (US); Richard Siemen, Shelby Township, MI (US)

(21) Appl. No.: 18/498,172

(22) Filed: Oct. 31, 2023

(51) Int. Cl. G01N 33/207; G06F 30/23; G06F 119/18

(52) U.S. Cl. CPC G01N 33/207; G06F 30/23; G06F 2119/18

(57) ABSTRACT

A method for estimating the strength of a laser welded blank including a first blank and a second blank joined together by a weld seam. The method may include forming a plurality of indentations in the first blank, the second blank, and the weld seam to determine an average value of a micro-hardness (Vickers); determining a scale-up ratio K; multiplying the scale-up ratio K by the average values of the micro-hardness of each of the first blank, the second blank, and the weld seam to obtain a scaled-up average value of the microhardness of each of the first blank, the second blank, and the weld seam; running a finite elemental analysis (FEA) simulation using the scaled-up average values of the microhardness; and based on the results of the simulation, determining whether the strength of the laser welded blank is sufficient to withstand being subjected to a forming process.

### FIELD

The present disclosure relates to a method for determining the strength of a weld seam of a laser welded blank.

### BACKGROUND

This section provides background information related to the present disclosure which is not necessarily prior art.

A laser welded blank (LWB) is formed by laser welding a first blank to a second blank, and has been used in the automotive industry for a number of years to form vehicle body components. The materials selected for each of the first blank and the second blank can be the same, or the materials selected for reach of the first blank and the second blank can be different. For example, the first blank can be formed of one steel grade and the second blank can be formed of a second steel grade. In addition, each grade can have a different thickness and/or include a coating (e.g., anti-corrosion coating). In this manner, after the LWB is stamped, the correct material may be positioned on the vehicle in the correct location to ensure greater safety and performance.

When different materials are used for each of the first and second blanks, it can be difficult to determine the strength of the weld seam between the first and second blanks. In this regard, the weld seam can be very narrow (e.g., 1 mm to 2 mm in width); the molten material that is formed during the laser welding can cool very quickly, which can affect the strength of the weld seam; and the different materials of the first and second blanks intermix during the laser welding, which creates a material having properties (e.g., hardness, tensile strength, etc.) that are different from the properties of the first and second blanks. The only way to determine whether the weld seam will withstand the stamping process or withstand being subjected to different tensile strains (i.e., during a collision event) applied to the weld seam is by conducting physical tests on the weld seam.

Put another way, there is currently no way to simulate these physical tests using computer simulations to determine the efficacy of the weld seam. Because there is currently no way to simulate these physical tests on the weld seam itself, the existence of the weld seam between the first and second blanks is typically ignored when conducting simulations on a LWB formed of two different materials, which may lead to an incorrect conclusion on whether the LWB can withstand the strains that may be applied to the LWB during stamping or a collision event.

### SUMMARY

This section provides a general summary of the disclosure, and is not a comprehensive disclosure of its full scope or all of its features.

The present disclosure provides a method for estimating the strength of a laser welded blank including a first blank and a second blank joined together by a weld seam. The method may include forming a plurality of indentations in the first blank, the second blank, and the weld seam to determine an average value of a micro-hardness (Vickers) of each of the first blank, the second blank, and the weld seam; determining a scale-up ratio K by dividing the average value of the micro-hardness of the weld seam (H_{W}) by the average value of the micro-hardness of at least one of the first blank and the second blank (H_{B}); multiplying the scale-up ratio K by the average values of the micro-hardness of each of the first blank, the second blank, and the weld seam to obtain a scaled-up average value of the microhardness of each of the first blank, the second blank, and the weld seam; running a finite elemental analysis (FEA) simulation using the scaled-up average values of the microhardness; and based on the results of the simulation, determining whether the strength of the laser welded blank is sufficient to withstand being subjected to a forming process.

According to the method of the present disclosure, the first blank may be formed of a first material having a first thickness and the second blank may be formed of a second material having a second thickness.

According to the method of the present disclosure, the first material may be the same as the second material.

According to the method of the present disclosure, the first thickness of the first material may be either the same as or different from the second thickness of the second material.

According to the method of the present disclosure, the first material may be different from the second material, and the first thickness of the first material may be either the same as or different from the second thickness of the second material.

According to the method of the present disclosure, the running the FEA simulation includes determining maximum strains that occur in a direction along a length of the weld seam and determining maximum strains that occur in a direction normal to a length of the weld seam.

According to the method of the present disclosure, the method may also include physically testing the laser welded blank to determine an average strain experienced by the weld seam along the length of the weld seam and the average strain experienced by the weld seam in the direction normal to the length of the weld seam.

According to the method of the present disclosure, the method may also comparing the average strain experienced in the direction along the length of the weld seam relative to the maximum strain experienced in the direction along the length of the weld seam during the simulation, and comparing the average strain experienced in the direction normal to the length of the weld seam to the maximum strain experienced in the direction normal to the length of the weld seam during the simulation.

According to the method of the present disclosure, if the maximum strains are less than the average strains, determining that the strength of the laser welded blank is sufficient to withstand being subjected to a forming process.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates a laser welded blank manufactured according to a principle of the present disclosure;

FIG. 2 is a flow chart setting forth a method according to the present disclosure;

FIG. 3 illustrates hardness values determining during Charpy V-notch testing;

FIG. 4 is a screen shot of a software program that can simulate strains applied to a laser welded blank;

FIG. 5 is a screen shot of a software program that can simulate strains applied to a laser welded blank using a scaled-up average micro-hardness determined according to a principle of the present disclosure;

FIG. 6 schematically illustrates strains experienced by a laser welded blank in directions along a length of a weld seam between the blanks; and

FIG. 7 schematically illustrates strains experienced by a laser welded blank in directions normal to a length of a weld seam between the blanks.

Corresponding reference numerals indicate corresponding parts throughout the several views of the drawings.

### DETAILED DESCRIPTION

Example embodiments will now be described more fully with reference to the accompanying drawings. The example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

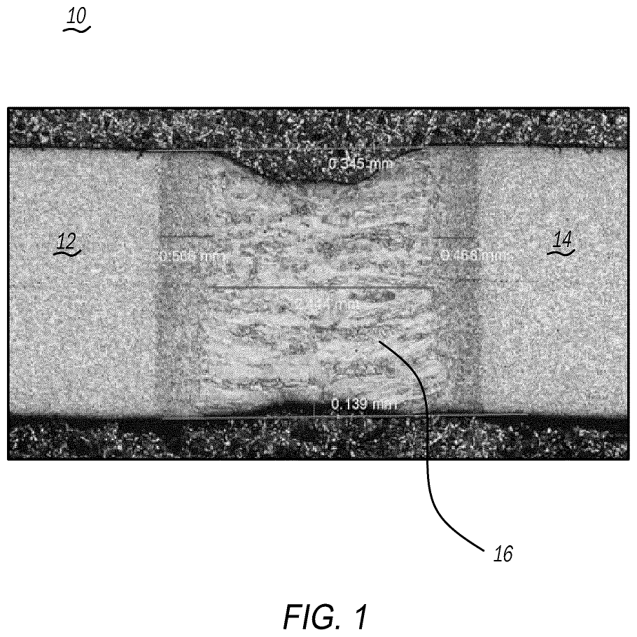

FIG. 1 illustrates a laser welded blank (LWB) 10 that includes a first blank 12, a second blank 14, and a weld seam 16 located between the first blank 12 and the second blank 14. The first and second blanks 12 and 14 can be formed of the same material or different materials, and the weld seam 16 is formed of a combination of the materials selected for each of the first and second blanks 14 and 16.

For example, first and second blanks 12 and 14 can each formed of steel; first and second blanks 12 and 14 can be formed of different grades of steel; first and second blanks 12 and 14 can each be formed of steel and one of the first and second blanks 12 and 14 includes a coating (e.g., a coating that includes zinc or some other type of coating known to one skilled in the art); one of the first and second blanks 12 can be formed of aluminum or some other type of metal material (e.g., titanium) and the other of the first and second blanks 12 and 14 can be formed of steel (coated or un-coated); first and second blanks 12 and 14 can be formed of different grades of aluminum (coated or uncoated); and first and second blanks 12 and 14 can have different thicknesses. This list is not exhaustive, and the primary aspect to keep in mind is that blanks 12 and 14 can be formed of any material known to one skilled in the art, and the materials selected for each of the first and second blanks 12 and 14 can affect the weld seam that joins the first and second blanks 12 and 14 together.

As noted above, because there is currently no way to simulate physical testing on the weld seam 16 itself, the existence of the weld seam 16 between the first and second blanks 12 and 14 is typically ignored when conducting simulations (e.g., finite elemental analysis (FEA)) on the LWB 10, which may lead to an incorrect conclusion on whether the LWB 10 can withstand the strains that may be applied to the LWB 10 during stamping or a collision event. More specifically, while it is possible to define material models for the materials of the first and second blanks 12 and 14, it is currently very difficult to define a material model for the weld seam 16, which is necessary to run a three-material simulation (i.e., the material of the first blank 12, the material of the second blank 14, and the material of the weld seam 16).

It is difficult to define a material model for the weld seam 16 because the weld seam 16 can be very narrow (e.g., 1 mm to 2 mm in width); the molten material that is formed during the laser welding can cool very quickly, which can affect the strength of the weld seam 16; and the different materials of the first and second blanks 12 and 14 intermix during the laser welding, which creates a weld seam 16 formed of a material having properties (e.g., hardness, tensile strength, etc.) that are different from the properties of the first and second blanks 12 and 14. In addition, there is a lack of data directed to weld seam formability or forming limits (i.e., how a weld seam 16 reacts when subjected to forming (e.g., stamping) or in a collision event). In the absence of these models and data, LWBs 10 may be formed that can fail when subjected to different strains, which may require the configuration of LWB 10 to be redesigned or a re-building of the stamping process and stamping dies associated therewith. In either case, money and time may be wasted.

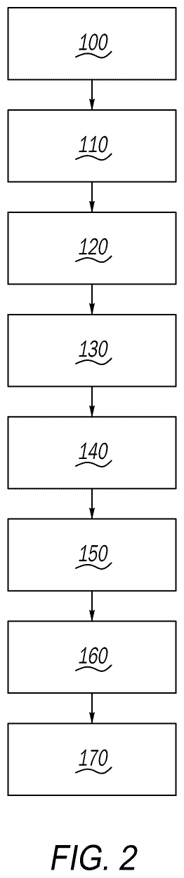

With the above in mind, the present disclosure provides a method for determining whether the weld seam 16 can withstand being subjected to strains that may be applied to the LWB 10 during stamping or a collision event. Referring to FIG. 2, the method includes a first step 100 of laser welding a plurality of the first blanks 12 to a plurality of the second blanks 14 to form a plurality of LWBs 10. In the example embodiment described below, in step 100 the first and second blanks 12 and 14 of each LWB 10 can be, for example, formed of the same material and same thickness (SM-ST), same material and different thickness (SM-DT), different materials and same thickness (DM-ST), and different materials and different thickness (DM-DT). As noted above, however, it will be understood and appreciated by one skilled in the art that at least one of the first and second blanks 12 and 14 may have a coating.

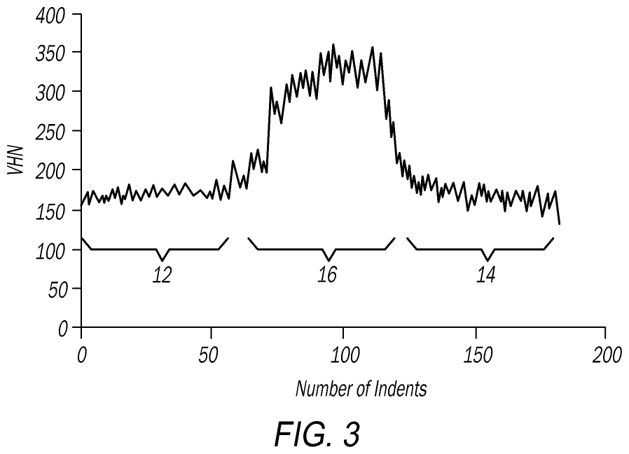

After the LWBs 10 are formed in step 100, an average valve of a micro-hardness (Vickers hardness) of each of the first blank 12, second blank 14, and weld seam 16 are measured, and the LWB 10 is subjected to strain testing along a length of the weld seam 16 and normal to the weld seam (step 110). The micro-hardness of the blanks 12, 14 and weld seam 16 is determined by conducting micro-hardness indentation testing over surfaces of the first blank 12, the second blank 14, and the weld seam 16. After conducting the indentation testing, the Vickers hardness numbers (VHN) may be graphed as shown in FIG. 3 where the hardness at each indentation is illustrated. FIG. 3 also exhibits the VHNs of the first blank 12, the second blank 14, and the weld seam 16.

In addition, the data associated with the average VHNs of the first blank 12, second blank 14, and weld seam 16 determined during the indentation testing can be input into a data base like that shown below in Table 1 (step 120).

TABLE 1

| | | | | DP | DP | DP |
|---|---|---|---|---|---|---|
| | Second | CR3 | CR340 | 780 | 980 | 1180 |
| | Blank 14 | T1.2 | T1.2 | T1.2 | T1.6 | T1.4 |
| First | Blank | 80 | 136-154 | 270 | 302-325 | 328-370 |
| Blank 12 | Hardness | | | | | |
| CR3 T1.2 | 80 | 154 | | | | |
| CR340 T1.2 | 136-154 | 307 | 349 | | | |
| CR340 T2.6 | 147 | | 316 | | | |
| DP 780 T1.2 | 270 | | | 422 | | |
| DP 980 T1.6 | 302-305 | | | | 444 | |
| DP 980 T1.3 | 314 | | | | 448 | |
| DP 1180 T1.4 | 328-370 | | | 479 | | 471 |

In the above Table 1, the different combinations of steels were used for the first and second blanks 12 and 14 that were laser welded together and then subjected to the indentation testing to determine an average Vickers micro-hardness of the blanks 12, 14 and weld seam 16. In this regard, steels of the same grade were used for the first and second blanks 12 and 14, steels of different grades were used for the first and second blanks 12 and 14, and steels of the same and different grades, but having different thicknesses, were used for the first and second blanks 12 and 14. For example, steel “CR3 T1.2” represents grade 3 cold rolled steel having a thickness of 1.2 mm, steel “CR 340 T1.2” represents high strength steel 340 having a thickness of 1.2 mm, “DP 780 T1.2” represents dual phase steel 780 having a thickness of 1.2 mm, and so on. The hardness of each of these blanks 12 and 14 is shown in Table 1, as well as the hardness of the weld seam 16.

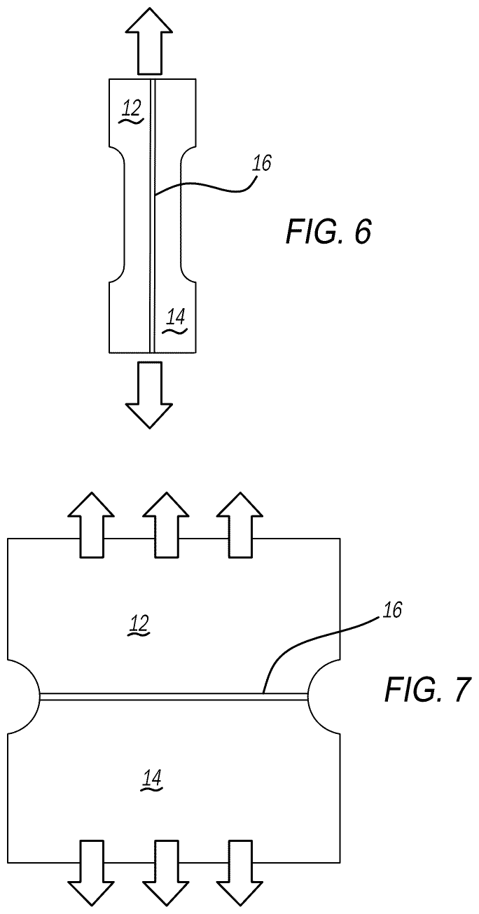

With respect to the strains experienced by the weld seam 16 along the length of the weld seam 16 and normal to the weld seam (see, e.g., FIGS. 6 and 7, respectively), the average strain values for each combination of blanks 12 and 14 can be tabulated as shown it the below Tables 3 and 4.

Next in step 130, a scale-up ratio K can be calculated, which is a ratio of a hardness (Hw) of the weld seam 16 to a hardness (Hb) of the material of the blank (i.e., K=(Hw/Hb)). After the scale-up ratio K is calculated, a hardening curve (stress-strain curve) similar to the one shown in FIG. 3 can be created by multiplying stress values of the material of the blank 12 or 14 (e.g., yield stress and/or tensile stress) indentation-by-indentation by K (step 140) to obtain a scaled-up hardness of each of the first blank 12, the second blank 14, and the weld seam 16. The stress values of the materials of the blanks 12, 14 are readily available. For example, when the first and second blanks 12 and 14 are each formed of grade 3 cold rolled steel having a thickness of 1.2 mm, the data from Table 1 can be used to determine K and this value can be multiplied by the yield stress and the tensile stresses of the blanks 12, 14 to yield an estimated yield stress and tensile stress of the weld seam 16. See the below Table 2, where the estimated yield stress and tensile stress of the weld seam 16 obtained are underlined. The data generated for the weld seam 16 creates an effective material model for the material of the weld seam 16, which due to the complexity of the microstructure and geometric features of the weld seam 16 is satisfactory at this time due to current experimental limitations for setting up a three-material (i.e., material of the blank 12, material of the blank 14, and material of the weld seam 16) simulation to more closely model the laser welded blank 10 including the first blank 12, second blank 14, and weld seam 16.

TABLE 2

| | Base Metal (CR3 T1.2) | Weld Seam |
|---|---|---|
| Yield Stress | 142.9 | 275.083 |
| Tensile Stress | 569.4 | 1096.095 |
| Weld Hardness | 154 | |
| (from Table 1) | | |
| Base Metal Hardness | 80 | |
| (from Table 1) | | |
| Scale-Up Ratio (K) | 1.925 | |

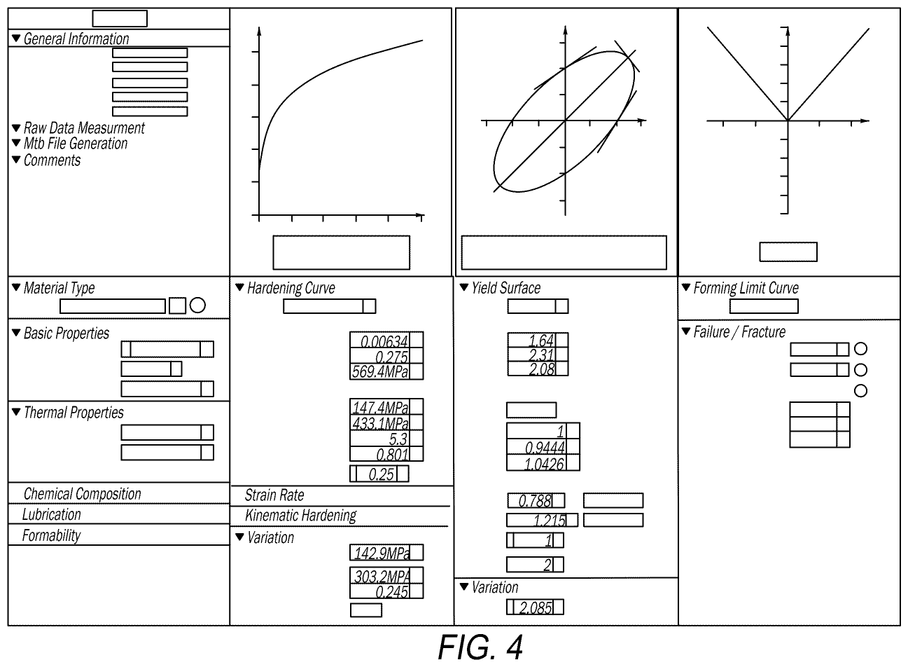

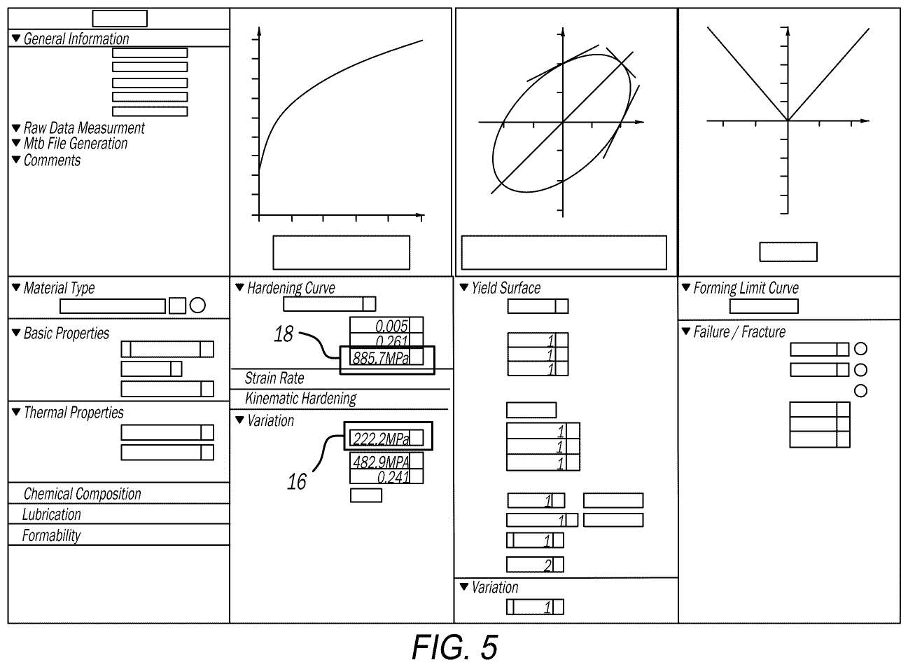

Next (step 150), a simulation can be generated using, for example, a FEA software with the three materials (i.e., material of the first blank 12, second blank 14, and weld seam 16). FIG. 4 illustrates various data that can be entered into the software relative to the blanks 12, 14, and FIG. 5 illustrates the various data that can be entered into the software relative to weld seam 16. When referring to FIG. 5, it should be noted that the weld seam 16 estimated yield stress and tensile stress listed in Table 2 was used (shown in boxes 18 and 20). While not shown in FIGS. 4 and 5, it should be understood that the thicknesses of the blanks 12 and 14 and weld seam 16 can also be input into the FEA software. It may be important to include the thicknesses of the blanks 12 and 14 and weld seam 16 because, as shown in FIG. 1, a thickness of the weld seam 16 can be less than a thickness of the blanks 12 and 14. The reduced thickness of weld seam 16 can affect the integrity of the laser welded blank 10 and, therefore, it may be important to measure and input the thickness of the weld seam 16 into the FEA software program.

After running the FEA simulation using the FEA software, data can be obtained relative to the major strains experienced along the weld seam 16 (see, e.g., FIG. 6) and normal to the weld seam 16 (see, e.g., FIG. 7). This data can then be tabulated (step 160) for comparison with the average strains calculated during physical strain testing of the LWB 10 and listed in the below Tables 3 and 4, which shows the average strain along a length of the weld seam 16 (Table 3) and normal to the weld seam 16 (Table 4) for various combinations of blanks 12 and 14.

TABLE 3

| | Strain Along Weld Seam | | | | | |
|---|---|---|---|---|---|---|
| | Second | | | DP | DP | DP |
| | Substrate | CR3 | CR340 | 780 | 980 | 1180 |
| | 14 | T1.2 | T1.2 | T1.2 | T1.6 | T1.4 |
| | First Substrate 12 | | | | | |
| | Limit | | | | | |
| | Major | | | | | |
| | Strain | 0.307 | 0.176 | 0.122 | 0.087 | 0.078 |
| CR3 T1.2 | 0.307 | 0.28 | | | | |
| CR340 T1.2 | 0.176 | 0.194 | 0.137 | | | |
| CR340 T2.6 | 0.195 | | 0.162 | | | |
| DP 780 T1.2 | 0.122 | | | 0.086 | | |
| DP 980 T1.6 | 0.087 | | | | 0.08 | |
| DP 980 T1.3 | 0.104 | | | | 0.057 | |
| DP 1180 T1.4 | 0.078 | | | 0.064 | | 0.058 |

TABLE 4

| | Strain Limit Normal to Weld Seam | | | | | |
|---|---|---|---|---|---|---|
| | Second | | | DP | DP | DP |
| | Substrate | CR3 | CR340 | 780 | 980 | 1180 |
| | 14 | T1.2 | T1.2 | T1.2 | T1.6 | T1.4 |
| | First Substrate 12 | | | | | |
| | Limit | | | | | |
| | Major | | | | | |
| | Strain | 0.307 | 0.176 | 0.122 | 0.087 | 0.078 |
| CR3 T1.2 | 0.307 | 0.171 | | | | |
| CR340 T1.2 | 0.176 | 0.024 | 0.056 | | | |
| CR340 T2.6 | 0.195 | | 0.018 | | | |
| DP 780 T1.2 | 0.122 | | | 0.05 | | |
| DP 980 T1.6 | 0.087 | | | | 0.038 | |
| DP 980 T1.3 | 0.104 | | | | 0.025 | |
| DP 1180 T1.4 | 0.078 | | | 0.019 | | 0.027 |

After running the simulations and tabulating the data associated with the average major strains along and normal to the weld seam 16, the strength of LWB 10 (i.e., weld seam 16) can be evaluated in step 170 to determine whether LWB 10 can withstand the stamping process or withstand being subjected to different tensile strains (i.e., during a collision event). This is done by reviewing the data obtained from the simulations and finding the maximum major strain along the weld seam 16 (i.e., in the direction shown in FIG. 6) and normal to the weld 16 (i.e., in the direction shown in FIG. 7) comparing the maximum major strains to the corresponding average major strain values listed in Table 3 and 4. For example, if the first and second blanks 12 and 14 are formed of grade 3 cold rolled steel having a thickness of 1.2 mm, the maximum major strain values determined during the FEA simulation along the weld seam 16 and normal to the weld seam 16 need to be less than 0.28 and 0.171, respectively (i.e., the values in BOLD in Tables 3 and 4). If each value is less than the values listed in Tables 3 and 4, the LWB 10 is considered satisfactory to withstand strains experienced during a stamping process or withstanding being subjected to different tensile strains (i.e., during a collision event).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for estimating the strength of a laser welded blank including a first blank and a second blank joined together by a weld seam, the method comprising:
forming a plurality of notches in the first blank, the second blank, and the weld seam to determine an average value of a micro-hardness (Vickers) of each of the first blank, the second blank, and the weld seam;
determining a scale-up ratio K by dividing the average value of the micro-hardness of the weld seam (H_{W}) by the average value of the micro-hardness of at least one of the first blank and the second blank (H_{B});
multiplying the scale-up ratio K by the average values of the micro-hardness of each of the first blank, the second blank, and the weld seam to obtain a scaled-up average value of the microhardness of each of the first blank, the second blank, and the weld seam;
running a finite elemental analysis (FEA) simulation using the scaled-up average values of the microhardness; and
based on the results of the simulation, determining whether the strength of the laser welded blank is sufficient to withstand being subjected to a forming process.

2. The method according to claim 1, wherein the first blank is formed of a first material having a first thickness and the second blank is formed of a second material having a second thickness.

3. The method according to claim 2, wherein the first material is the same as the second material.

4. The method according to claim 3, wherein the first thickness of the first material is either the same as or different from the second thickness of the second material.

5. The method according to claim 2, wherein the first material is different from the second material.

6. The method according to claim 5, wherein the first thickness of the first material is either the same as or different from the second thickness of the second material.

7. The method according to claim 1, wherein the running the FEA simulation includes determining maximum strains that occur in a direction along a length of the weld seam and determining maximum strains that occur in a direction normal to a length of the weld seam.

8. The method according to claim 7, further comprising physically testing the laser welded blank to determine an average strain experienced by the weld seam along the length of the weld seam and the average strain experienced by the weld seam in the direction normal to the length of the weld seam.

9. The method according to claim 8, further comprising comparing the average strain experienced in the direction along the length of the weld seam relative to the maximum strain experienced in the direction along the length of the weld seam during the simulation, and comparing the average strain experienced in the direction normal to the length of the weld seam to the maximum strain experienced in the direction normal to the length of the weld seam during the simulation.

10. The method according to claim 9, wherein if the maximum strains are less than the average strains, determining that the strength of the laser welded blank is sufficient to withstand being subjected to a forming process.
